(19) United States
(12) Patent Application Publication
(10) Pub. No.: US 2008/0014179 A1
(43) Pub. Date: Jan. 17, 2008

(54) TISSUE TRANSPLANTATION COMPOSITIONS AND METHODS

(76) Inventor: Bret A. Ferree, Cincinnati, OH (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/775,639

(22) Filed: Jul. 10, 2007

Related U.S. Application Data
(60) Provisional application No. 60/830,009, filed on Jul. 11, 2006.
(60) Provisional application No. 60/829,970, filed on Oct. 18, 2006.

(51) Int. Cl. A61K 35/12; A61F 2/02; A61K 35/30; A61K 48/00; A61P 43/00; A61K 35/32; A61F 2/44

(52) U.S. Cl. 424/93.7; 128/898; 424/520; 424/570; 424/572; 623/14.12; 623/17.11; 623/23.72

(57) ABSTRACT

A biomedical material for transplant to a subject is provided according to embodiments of the present invention which includes an isolated donor tissue enzyme-treated to reduce the amount of proteoglycans in the donor tissue compared to untreated tissue. Isolated cells are optionally added to the enzyme-treated donor tissue, including leukocytes, particularly monocytes; macrophages; platelets; cells derived from an intervertebral disc such as chondrocyte-like nucleus pulposus cells; fibrocytes; fibroblasts; mesenchymal stem cells; mesenchymal precursor cells; chondrocytes; or a combination of any of these. The isolated donor tissue is articular cartilage or an intervertebral disc tissue such as nucleus pulposus tissue and/or annulus fibrosis tissue enzyme-treated to remove proteoglycans normally present in these tissues. A biomedical material of the present invention is administered to a subject to treat a disorder or injury, such as a disorder or injury to connective tissue.

### REFERENCE TO RELATED APPLICATIONS

This application claims priority from U.S. Provisional Patent Application Ser. Nos. 60/830,009, filed Jul. 11, 2006, and 60/829,970, filed Oct. 18, 2006, the entire content of both of which is incorporated herein by reference.

### FIELD OF THE INVENTION

The present invention relates generally to methods and compositions for transplant treatment of a subject. In specific embodiments, the present invention relates to methods and compositions for transplant treatment of a connective tissue disorder and/or injury in a subject.

### BACKGROUND OF THE INVENTION

Connective tissues, particularly articular cartilage and intervetebral discs have limited healing properties. Unfortunately, injury and disease affecting articular cartilage or intervetebral discs are among the most common chronic conditions.

For example, premature or accelerated disc degeneration is known as degenerative disc disease. A large portion of patients suffering from chronic low back pain are thought to have this condition. As the disc degenerates, the nucleus and annulus functions are compromised. The nucleus becomes thinner and less able to handle compression loads. The annulus fibers become redundant as the nucleus shrinks. The redundant annular fibers are less effective in controlling vertebral motion. The disc pathology can result in: 1) bulging of the annulus into the spinal cord or nerves; 2) narrowing of the space between the vertebra where the nerves exit; 3) tears of the annulus as abnormal loads are transmitted to the annulus and the annulus is subjected to excessive motion between vertebra; and 4) disc herniation or extrusion of the nucleus through complete annular tears.

Current surgical treatments of disc degeneration are destructive. One group of procedures removes the nucleus or a portion of the nucleus; lumbar discectomy falls in this category. A second group of procedures destroy nuclear material; Chymopapin (an enzyme) injection, laser discectomy, and thermal therapy (heat treatment to denature proteins) fall in this category. A third group, spinal fusion procedures, either remove the disc or the disc's function by connecting two or more vertebra together with bone. These destructive procedures lead to acceleration of disc degeneration. The first two groups of procedures compromise the treated disc. Fusion procedures transmit additional stress to the adjacent discs. The additional stress results in premature disc degeneration of the adjacent discs.

Prosthetic disc replacement offers many advantages. The prosthetic disc attempts to eliminate a patient's pain while preserving the disc's function. Current prosthetic disc implants, however, either replace the nucleus or the nucleus and the annulus. Both types of current procedures remove the degenerated disc component to allow room for the prosthetic component.

Several hundred thousand patients undergo disc operations each year. Approximately five percent of these patients will suffer recurrent disc herniation, which results from a void or defect which remains in the outer layer (annulus fibrosis) of the disc after surgery involving partial discectomy. The defect acts as a pathway for additional material to protrude into the nerve, resulting in the recurrence of the herniation. This results in pain and further complications, in many cases.

Apart from destructive techniques, patients with herniated intervertebral discs and degenerative disc disease may conservatively be treated by rest, physical therapy, oral medication, and chiropractic care. Patients that do not respond to conservative care generally undergo an injection of steroids into the epidural space of their spinal canal (epidural space) or surgery. Steroid injection reduces the inflammation surrounding herniated or degenerated discs. Decreased inflammation may reduce the pain from the disc. Unfortunately, steroid injection may hinder the healing process. Although growth factors and differentiation factors (soluble regulators) induce the healing process, it is believed that steroids may interfere with the cascade of these healing factors normally found in the body.

Similarly, articular cartilage disease or injury is often difficult to treat satisfactorily.

Over 7,000 patients have been treated with a procedure known as Autologous Chondrocyte Transplantation (ACT). The procedure involves removing a piece of articular cartilage from a non-weight bearing portion of a patient's knee, releasing the cartilage cells from the autograft tissue, and culturing the cells to expand the cell population 20-50 fold. The autologous cartilage cells can be used to treat defects in patients' articular cartilage. ACT may generate normal articular cartilage known as hyaline cartilage. Unfortunately, the procedure may generate a less desirable type of cartilage known as fibrocartilage.

My issued U.S. Pat. Nos. 6,340,369; 6,344,058; 6,352,557; 6,419,702; 6,645,247; 6,648,918; 6,648,919; 6,648,920; 6,454,804; 6,685,695; 6,793,677 & 6,878,167 and pending U.S. patent application Ser. Nos. 10/526,993; 10/876,792; 10/853,296; 10/853,443; 10/303,385; 10/864,160 teach tissue engineering to treat diseases of the intervertebral disc, the content of each being expressly incorporated herein by reference in their entirety.

However, there is a continuing need for methods and compositions for transplant treatment of disorders and/or injuries to intervertebral discs and/or articular cartilage in a subject.

### SUMMARY OF THE INVENTION

A biomedical material is provided according to embodiments of the present invention which includes an enzyme-treated isolated donor tissue, the enzyme-treated donor tissue characterized by a reduced amount of at least one type of proteoglycan compared to untreated tissue. In particular embodiments of the present invention, the biomedical material further includes a quantity of isolated cells in contact with the enzyme-treated isolated donor tissue. The isolated cells are leukocytes, particularly monocytes; macrophages; platelets; cells derived from an intervertebral disc such as chondrocyte-like nucleus pulposus cells; fibrocytes; fibroblasts; mesenchymal stem cells; mesenchymal precursor cells; chondrocytes; or a combination of any of these in certain embodiments.

The enzyme-treated isolated donor tissue is optionally a human or non-human tissue. In particular embodiments, the enzyme-treated isolated donor tissue is connective tissue. In further particular embodiments, the enzyme-treated isolated donor tissue is articular cartilage or an intervertebral disc tissue such as nucleus pulposus tissue and/or annulus fibrosis tissue.

An enzyme-treated isolated donor tissue is optionally treated to render it substantially free of intact, living, cells endogenous to the enzyme-treated isolated donor tissue. Thus, for example, cells present in the donor tissue when the tissue is isolated from its natural environment are killed prior to use as a biomedical material in particular embodiments.

Enzyme treatment of the isolated results in reduction in the amount of at least one type of proteoglycan by 1-100% compared to an untreated tissue of the same type.

In certain embodiments, the quantity of isolated cells included in a biomedical material for transplant according to the present invention is in the range of about 10³-10⁹ cells/milliliter of the enzyme-treated donor tissue.

The enzyme-treated donor tissue is processed to form particles in particular embodiments of a biomedical material of the present invention. For example, the particles may have an average particle size in the range of about 0.01 mm³-30 mm³, inclusive.

A method of treating a defective tissue in a subject is provided according to embodiments of the present invention which includes introducing a biomedical material which includes an enzyme-treated isolated donor tissue, the enzyme-treated donor tissue characterized by a reduced amount of at least one type of proteoglycan compared to untreated tissue into a subject having a defective tissue.

In particular embodiments, the defective tissue is an intervertebral disc or articular cartilage.

The biomedical material is introduced into the subject in or near a region of the defective tissue in preferred embodiments of an inventive method.

A method of treating a defective tissue in a subject according to further specific embodiments of the present invention includes introducing a biomedical material which includes an enzyme-treated isolated donor tissue characterized by a reduced amount of at least one type of proteoglycan compared to untreated tissue admixed with a quantity of isolated cells into a subject having a defective tissue. The isolated cells are leukocytes, particularly monocytes; macrophages; platelets; cells derived from an intervertebral disc such as chondrocyte-like nucleus pulposus cells; fibrocytes; fibroblasts; mesenchymal stem cells; mesenchymal precursor cells; chondrocytes; or a combination of any of these in certain embodiments.

The quantity of isolated cells is isolated from the subject, from an individual of the same species as the subject and/or from an individual of a different species than the subject.

Optionally, the quantity of isolated cells is characterized as having a genotype identical to a genotype of the subject.

In particular embodiments of a method of the present invention, isolated cells to be admixed with an enzyme-treated isolated donor tissue characterized by a reduced amount of at least one type of proteoglycan compared to untreated tissue are expanded in vitro following isolation of the cells and prior to admixture with the tissue.

In a further option, the quantity of isolated cells admixed with an enzyme-treated isolated donor tissue characterized by a reduced amount of at least one type of proteoglycan compared to untreated tissue includes cells pooled following isolation from the subject, one or more individuals of the same species as the subject and/or one or more individuals of a different species compared to the subject.

In a particular embodiment of the present invention, the enzyme-treated donor tissue is incubated with the quantity of isolated cells for a period of time prior to introducing the biomedical material into the subject.

A commercial package for treatment of a defective tissue in a subject is provided according to embodiments of the present invention which includes a quantity of a proteoglycan-cleaving enzyme; and a quantity of isolated cells selected from leukocytes, particularly monocytes; macrophages; platelets; cells derived from an intervertebral disc such as chondrocyte-like nucleus pulposus cells; fibrocytes; fibroblasts; mesenchymal stem cells; mesenchymal precursor cells; chondrocytes; or a combination of any of these. Optionally, a culture medium suitable for growth and/or maintenance of the quantity of isolated cells is included in the commercial package.

In a further embodiment, a commercial package for treatment of a defective tissue in a subject includes an enzyme-modified isolated donor tissue. For example, the enzyme-modified isolated donor tissue is enzyme-modified isolated donor connective tissue.

Optionally, a quantity of cells such as leukocytes, particularly monocytes; macrophages; platelets; cells derived from an intervertebral disc such as chondrocyte-like nucleus pulposus cells; fibrocytes; fibroblasts; mesenchymal stem cells; mesenchymal precursor cells; chondrocytes; or a combination of any of these, is included in the commercial package, for instance admixed with the enzyme-modified isolated donor tissue or in a container for later admixture with the enzyme-modified isolated donor tissue. Physicians could add cells or cells plus other materials, such as platelet rich plasma, to the enzyme-treated donor tissue. Thus, in one embodiment of a commercial package an additional material, such as platelet rich plasma is included, for instance admixed with the enzyme-modified isolated donor tissue or in a container for later admixture with the enzyme-modified isolated donor tissue. Further additional materials

A method of tissue transplantation is provided according to embodiments of the present invention which includes providing tissue to be transplanted; using an enzyme to at least partially digest proteoglycans in the tissue while at least partially preserving the collagen network of the tissue; and transplanting the treated tissue into a recipient. Optionally, the provided tissue to be transplanted is an allograft tissue, a xenograft tissue, and/or autograft tissue. In particular embodiments of a method of the present invention, the tissue to be transplanted is derived from, or forms part of, articular cartilage and/or an intervertebral disc.

In particular embodiments of a method of the present invention, the tissue to be transplanted is treated with a proteoglycan-cleaving enzyme to reduce the proteoglycan content of the tissue by 1-100% compared to an untreated tissue. Optionally, stem cells are added to the tissue. In specific embodiments, the tissue is connective tissue.

A method according to embodiments of the present invention is a method in which transplanted tissue is used to heal tears or clefts within an intervertebral disc structure such as a nucleus pulposus; an annulus fibrosus; or a combination thereof.

In further embodiments of a method of the present invention, the transplanted tissue is used to increase the volume of an intervertebral disc structure such as a nucleus pulposus; an annulus fibrosus; or a combination thereof.

The tissue to be transplanted is treated with a proteoglycan-cleaving enzyme. In particular embodiments the proteoglycan-cleaving enzyme is an enzyme which digests polysaccharide side chains in a proteoglycan without digesting the protein portion of the proteoglycan. Proteoglycan-cleaving enzymes include such enzymes as chondroitinases, hyaluronidases and keratanases. Thus, proteoglycan-cleaving enzymes used to treat an isolated donor tissue to reduce proteoglycan content therein may be a chondroitinase, a hyaluronidase, a keratanase or a combination of these. In specific embodiments, the isolated donor tissue is treated with chondroitinase ABC, keratanase I, keratanase II, hyaluronidase, or a combination of these enzymes to reduce proteoglycan content of the tissue.

Additionally, a tissue to be transplanted is optionally treated with a protease such as chymopapain, collagenase, cathepsin B, cathepsin G, calpain I, or a combination of any of these. The tissue is then optionally treated with a second material that deactivates the enzyme.

In particular embodiments, the tissue to be transplanted is treated with alpha-2-macroglobulin or a cathepsin before transplanting the treated tissue into a recipient.

### BRIEF DESCRIPTION OF THE DRAWINGS

FIG. 1 shows a graph illustrating that sulfated proteoglycan concentration varied linearly with absorbance;

FIG. 2 shows a graph of the effect of proteoglycan-cleaving enzyme incubation with intervetebral disc tissue on proteoglycan concentration;

FIG. 3 shows a graph of the results of the treatment of human intervertebral disc particles with a combination of Chondroitinase ABC and Keratanase; and

FIG. 4 shows a graph of the results of proteoglycan quantitation following treatment of.

### DETAILED DESCRIPTION OF THE INVENTION

A biomedical material is provided herein which includes an isolated donor tissue processed so as to have a reduced amount of at least one type of proteoglycan compared to untreated tissue.

The term “enzye-treated donor tissue” is used herein to refer to an isolated donor tissue treated with one or more proteoglycan cleaving enzymes so as to have a reduced amount of at least one type of proteoglycan compared to untreated tissue donor tissue

A biomedical material according to the present invention is useful in treatment of injuries and diseases involving a defective tissue, particularly a defective connective tissue, in a subject.

Embodiments of the present invention include the use of one or more enzymes to at least partially digest one or more types of proteoglycans in isolated donor tissue to produce a modified connective tissue characterized by a reduced amount of at least one type of proteoglycan compared to untreated tissue for transplant to a subject. Removing proteoglycans creates spaces within the treated tissue for the attachment of cells. Removing proteoglycans in the isolated donor tissue improves the binding of cells to the modified connective tissue material, such as exogenously added and/or cells endogenous to the subject at or near the site of the transplant. Treatment of isolated donor tissue to at least partially remove proteoglycans also reduces the volume of transplanted modified connective tissue material while preserving the collagen network of the tissue. Furthermore, removing proteoglycans exposes proteins such as cytokines that facilitate binding of transplanted cells to the tissue. The exposed cytokines also attract cells in the subject to the transplanted tissue. The enzyme-treated tissue provides spatial and regulatory information to direct the cells that are added to the material or that migrate into the treated tissue, to express genes that cause the cells to assume the phenotype of the cells that were native to the enzyme-treated tissue. For example, such information could be used to direct mesenchymal stems cells to differentiate into chondrocyte-like cells that produce proteoglycan rich extracellular matrix.

In particular embodiments, the isolated donor tissue treated with an enzyme to remove at least a portion of the proteoglycans therein is intervertebral disc tissue, such as nucleus pulposus and/or annulus fibrosus; or articular cartilage.

Donor tissue is isolated from the subject to be treated, from one or more individuals of the same species as the subject, or from one or more individuals of a species other than that of the subject to be treated. In preferred embodiments, a donor tissue is isolated from an individual of a species other than that of the subject to be treated, such that the subject receiving the donor tissue receives a xenograft. In further embodiments, a donor tissue is isolated from an individual of the same species as the subject to be treated, such that the subject receiving the donor tissue receives an allograft. In still further embodiments, a donor tissue is isolated from the subject to be treated, such that the subject receiving the donor tissue receives an autograft.

Although the compositions and methods detailed herein are described primarily with reference to a human subject having a condition to be treated such as a connective tissue disorder and/or injury, it is appreciated that a subject to be treated may also be a non-human animal such as a non-human primate, dog, cat, horse or cow. Thus, for example, where a human is the subject to be treated, connective tissue is isolated from the subject, from another human individual or individuals, and/or from a non-human animal. Similarly, for example, where a dog is the subject to be treated, connective tissue is isolated from the subject, from another individual dog or dogs, and/or from a non-canine species.

Connective tissue is isolated according to methods known in the art, such as by surgical harvest of connective tissue from a living or cadaver human or non-human animal.

Following isolation of connective tissue, the tissue is optionally stored for later processing. For example, isolated donor tissue is stored at −70° C.

In a preferred option, isolated donor tissue is morselized to produce isolated donor tissue particles prior to treatment of the tissue to remove of at least some of the proteoglycans in the isolated donor tissue. The isolated donor tissue is morselized to enable insertion of the treated tissue into a patient's body through an injection or through a small incision. Morselization of the isolated donor tissue also increases the surface area of the tissue.

The size and shape of the particles depends on the application. For example, isolated donor tissue is morselized into particles having an average particle size in the range of about 0.01 mm³-30 mm³ in particular embodiments. In particular examples, isolated nucleus pulposus tissue is morselized into particles of approximately 1×1 mm in size or smaller on one surface, 2×2 mm, 3×3 mm, or 4×4 mm in size or larger on one surface. The pieces of isolated donor tissue can be approximately spherical or other shapes. In further embodiments, the connective tissue is articular cartilage and the cartilage may be cut into sizes and shapes to match defects in a patient's joints. For example, the cartilage could be morselized into particles having approximately circular shapes having an average diameter in the range of about 1-30 millimeters in diameter. In specific embodiments, the cartilage could be morselized into particles having approximately circular shapes and characterized by an average diameter of 1, 2, 3, 4, 5, 6, 7, 8, 9, 10, 12, 14, 16, 18, 20, 22, 24, 26, 28, 30 or more millimeters.

Morselization of isolated donor tissue may be achieved by cutting, such as with surgical scissors. In further embodiments, isolated donor tissue is frozen, such as by freezing in liquid nitrogen and the frozen tissue is morselized by grinding, for instance by use of a motorized grinding apparatus or by hand using a mortar and pestle. The size of the particles may be measured by standard particle measurement techniques such as by standard sieve, microscopy, comparison with a known particle size and by direct measurement.

Treatment of isolated donor tissue to reduce the amount of intact proteoglycan in the tissue includes incubation of the isolated donor tissue with a proteoglycan-cleaving enzyme to yield an enzyme-treated donor tissue. In particular embodiments the proteoglycan-cleaving enzyme is an enzyme which digests polysaccharide side chains in a proteoglycan without digesting the protein portion of the proteoglycan. Proteoglycan-cleaving enzymes are known in the art and include chondroitinases, keratanases and hyaluronidases. Combinations of these enzymes may also be used.

Particular examples of proteoglycan-cleaving enzymes include, but are not limited to, chondroitinase-ABC, keratanase I, keratanase II, and hyaluronidase.

An isolated donor tissue is treated to at least partially remove the proteoglycans by incubation of isolated donor tissue with one or more proteoglycan-cleaving enzymes.

For example, the morselized isolated nucleus pulposus tissue is treated with chondroitinase-ABC (0.25 IU/ml for 90 minutes at 37 degrees C.) and/or hyaluronidase (3,500 units/mI for thirty minutes) in particular embodiments. In a further exemplary embodiment, the morselized isolated nucleus pulposus tissue is treated with chondroitinase-ABC (0.1 units/ml, ICN Biochemicals), keratanase I (0.1 units/ml, Sigma), and keratanse II (0.001 units/ml, AMS Biotechnology, Whitney, UK) for three hours at room temperature. The particles of isolated nucleus pulposus tissue should be completely submerged in the enzymatic solutions.

The amount of enzyme used, the incubation time and the incubation temperature may be varied to achieve reduction of proteoglycan in the morselized isolated donor tissue.

For example, the morselized isolated nucleus pulposus tissue could be treated with a concentration of chondroitinase-ABC in the range of about 0.0001 U/ml-2 U/ml for a time in the range of about 30 minutes-12 hours. In particular examples, chondroitinase-ABC concentrations of about 1 IU/ml, 0.5 IU/ml, 0.25 IU/ml, 0.1 IU/ml, 0.05 IU/ml, 0.01 IU/ml, 0.005 IU/ml, 0.001 IU/ml, 0.005 IU/ml, or 0.0001 IU/ml or less are used for 30 minutes, 60 minutes, 2 hours, 3 hours, 4, hours, 5 hours, 6 hours, 7 hours, 8 hours, 10 hours, or 12 hours, or more.

In a further example, the morselized isolated nucleus pulposus tissue could be treated with a concentration of keratanase I in the range of about 0.0001 U/ml-1 U/ml for a time in the range of about 30 minutes-12 hours. In particular examples, keratanase I concentrations of about 1.0 unit/ml, 0.5 units/ml, 0.4 units/ml, 0.3 units/ml, 0.2 units/ml, 0.08 units/ml, 0.06 units/ml, 0.04 units/ml, 0.02 units/ml, 0.01 units/ml, 0.005 units/ml, 0.001 units/ml, 0.0005 units/ml, or 0.0001 units/ml are used for 30 minutes, 60 minutes, 2 hours, 3 hours, 4, hours, 5 hours, 6 hours, 7 hours, 8 hours, 10 hours, or 12 hours, or more.

In another example, the morselized isolated nucleus pulposus tissue could be treated with a concentration of keratanase II in the range of about 0.00001 units/ml-0.01 units/ml for a time in the range of about 30 minutes-12 hours. In particular examples, keratanase II concentrations of about 0.01 units/ml, 0.008 units/ml, 0.006 units/ml, 0.004 units/ml, 0.002 units/ml, 0.0008 units/ml, 0.0006 units/ml, 0.0004 units/ml, 0.0002 units/ml, or 0.00001 units/ml or less are used for 30 minutes, 60 minutes, 2 hours, 3 hours, 4, hours, 5 hours, 6 hours, 7 hours, 8 hours, 10 hours, or 12 hours, or more.

Combinations of any of chondroitinase-ABC, keratanase I and keratanase II may be used to remove proteoglycans from an isolated donor tissue. Thus, for example, a combination of any of: chondroitinase-ABC having a concentration in the range of about 0.0001 U/ml-1 U/ml; keratanase I having a concentration in the range of about 0.0001 U/ml-1 U/ml and iceratanase II having a concentration in the range of about 0.00001 units/ml-0.01 units/ml is used in a particular embodiment.

The amount of proteoglycan in an isolated donor tissue is reduced by 1-100% compared to an untreated connective tissue using a proteoglycan-cleaving enzyme. The extent of reduction of proteoglycan in isolated donor tissue is assessed by any of various assays known in the art illustratively including quantitation of proteoglycans using spectrophotometry as described in Whiteman, P., Biochem J., 1973, 131(2): 343-350 and herein.

The proteoglycan-cleaving enzyme-treated connective tissue is separated from released proteoglycans in particular embodiments of the present invention. For example, proteoglycan-cleaving enzyme-treated tissue is washed with phosphate buffered saline to remove the cellular debris, the proteoglycans that were released from the tissue, and the proteoglycan-cleaving enzymes. The proteoglycan-reduced tissue may also be separated from proteoglycans, fluids and/or debris with a centrifuge or a filter.

The enzyme-treated donor tissue may be frozen for storage.

A connective tissue is preferably harvested, processed, and stored under sterile conditions. In a further embodiment, the proteoglycan-reduced material can be sterilized before cells are added to the processed tissue. For example, the enzyme-treated donor tissue is sterilized with gamma radiation in a particular embodiment of the present invention. Materials could be added to the tissue to preserve the mechanical properties of the tissue during radiation treatment. Such materials are well known to those skilled in tissue banking techniques.

In particular embodiments of the present invention, the isolated donor tissue is treated so as to produce isolated donor tissue substantially free of intact, living, cells endogenous to the connective tissue. The cells within the isolated donor tissue are killed to reduce the risk of disease transmission and to reduce the risk of immune reaction. Treatment of tissue to produce a substantially acellular material includes disruption of cell membranes in the isolated donor tissue so as to rupture cells.

Further preferred is a treatment to produce a substantially acellular material without substantially denaturing most of the proteins in the tissue. Such methods are identified by assay of activity of an indicator protein, such as an enzyme, in the isolated donor tissue to evaluate activity quantitatively or qualitatively compared to an untreated tissue. An untreated tissue for purposes of comparison may be an untreated portion of a tissue from the same source as the treated portion or an untreated tissue of the same type from another source, for instance.

In one embodiment of the invention, isolated donor tissue is frozen at −70 degrees C. to kill the cells within the tissue.

In particular embodiments, the invention includes the use of other enzymes such as Chymopapain (0.1-50 mgs), Collagenase (400 ABC units), Cathepsins B and G, Calpain I, or other material may be used to treat the disc tissue. For example, disc particles less than 5 mm in diameter could be submerged in 1 ml of PBS and 4000 U chymopapain (or 3000 U, or 2000 U or 1000 U or less) for three hours (or two hours, or one hour, or less, or four hours, five hours or more). Larger disc particles could be treated with higher doses of chymopapain or longer processing times. The enzyme treated tissue may be treated with a second material that deactivates the enzyme. For example, in a method of treating a subject having an intervertebral disc disorder and/or injury, alpha-2-macroglobulin or cathepsins such as present in the subject's serum or tissues may be injected into the subject's disc after treating the disc with the enzyme but before injection of the therapeutic disc material. Similarly, the enzyme treated disc tissue can be treated with alpha-2-macroglobulin or cathepsins before injection of the material into a subject's disc.

The enzyme-treated donor tissue provides a scaffold for exogenously added cells and/or cells endogenous to the subject according to particular embodiments of the present invention. Transplant of the enzyme-treated donor tissue into a subject allows for rebuilding the proteoglycans within the transplanted tissue in vivo.

Thus, in particular embodiments, the biomedical material further includes a quantity of isolated cells in contact with the enzyme-treated donor tissue. A combination of enzyme-treated donor tissue and cells, is referred to herein interchangeably as “transplant material” or “therapeutic disc material” (TDM).

Cells admixed with enzyme-treated donor tissue are leukocytes, particularly monocytes; macrophages; platelets; cells derived from an intervertebral disc such as chondrocyte-like nucleus pulposus cells; fibrocytes; fibroblasts; mesenchymal stem cells; mesenchymal precursor cells; chondrocytes; or a combination of any of these in certain embodiments. Each of these types of cells is well-characterized and methods for their isolation, identification, culture, expansion and differentiation from an adult individual or as embryonic cells from various species, including humans, is known in the art. For instance, cells may be released from a tissue source by mechanical dispersion and/or enzymatic treatment followed by separation from cell debris, such as by centrifugation. Particular cells may be identified by morphology and/or by assay for the presence of cell type-specific markers known in the art. Detailed protocols for isolation and identification of particular cells are described, for example, in Lin, Z. et al., Tissue Eng., 2006, 12:1971-84; Bosnakovski, D. et al., Cell Tissue Res. 2005, 319(2):243-53; Schmitt, B. et al., Differentiation, 2003, 71(9-10):567-77; Steck, E. et al., Stem Cells, 2005, 23(3):403-11; Risbud, M. V., et al., Spine. 2004, 29(23):2627-32; Alhadlaq, A., and Mao, J. J., 2004, Stem Cells and Develop. 13: 436-448; Pittenger, M. F., and Marshak, D. R., in Marshak, D. R., et al., Eds., Stem Cell Biology, Cold Spring Harbor Laboratory Press, Cold Spring Harbor, N.Y., 2001; Pittenger, M. F., et al., 1999, 284: 143-147; and Prockop, D. I., 1997, Science 276: 71-74.

Cells, such as mesenchymal stem cells (MSCs), are added to the enzyme-treated donor tissue in a particular embodiment of the invention. The MSCs are preferably autologous MSCs.

For example, the isolated cells are bone marrow cells in particular embodiments. Bone marrow cells include mesenchymal stem cells and mononuclear cells among others. Bone marrow is harvested by methods known in the art, such as aspiration from the posterior iliac crest, sternum and/or anterior iliac crest.

Bone marrow cells are optionally applied to an enzyme-treated donor tissue without further purification from bone marrow. In further embodiments, particular bone marrow cells, such as mesenchymal stem cells and/or monocytes are purified from bone marrow.

In particularly preferred embodiments, mesenchymal stem cells are isolated from the subject to receive the transplant. The mesenchymal stem cells may be obtained from a bone marrow aspirate and/or from adipose tissue, for autologous use.

In one example, briefly described, isolation and expansion of mesenchymal stem cells is achieved essentially as described in Steck, E. et al., Stem Cells, 23:403-411, 2005, by isolation from bone marrow samples which are fractionated on a suitable density gradient, such as a FICOLL-PAQUE Plus density gradient available commercially from GE Healthcare, US, and the low density fraction enriched in mesenchymal stem cells is collected, and the cells are cultured in expansion medium in culture flasks. Exemplary expansion medium contains 2% fetal calf serum, recombinant human epidermal growth factor, recombinant human platelet-derived growth factor BB, 60% low-glucose DMEM (Gibco BRL), 40% MCDB-201 (Sigma), 1× insulin transferrin selenium, 1× linoleic acid bovine serum albumin (BSA), 10⁻⁹ M dexamethasone (Sigma), and 10⁻⁴ M ascorbic acid 2-phosphate (Sigma), 100 U penicillin, and 1000 U streptomycin (Gibco).

In a further example, the technique taught by Bruder S P et. al., J Bone and Joint Surg Am. 1998;80:985-96 could be used to isolate and expand mesenchymal stem cells.

Human mesenchymal stem cells are identified by particular markers illustratively including integrin beta-1 and ICAM-1 as well as negative markers CD45 and CD14. Further human mesenchymal cell markers are known in the art, as described in Pittenger, M. F., and Marshak, D. R., in Marshak, D. R., et al., Eds., Stem Cell Biology, Cold Spring Harbor Laboratory Press, Cold Spring Harbor, N.Y., 2001.

Methods for isolation, identification, culture, expansion of chondrocytes from animals of various species, including humans, are known in the art, such as described in M. K. Akens and M. B. Hurtig, BMC Musculoskelet. Disord., 2005, 6:23; and Barbero, A. et al., Osteoarthritis Cartilage, 2004, 12(6):476-84. For example, chondrocytes can be isolated as detailed in De Ceuninck, F., Arthritis Res Ther., 2004; 6(5): R393-R403. Briefly described, chondrocytes are isolated from cartilage by enzymatic digestion of the cartilage such as by incubation of cartilage in Ham F12 medium with 10% fetal calf serum in the presence of 3 mg/ml collagenase type I, Worthington, Lalcewood, N.J., and 2 mg/ml dispase from Bacillits polymixa, Invitrogen.

Chondrocyte markers illustratively include Sox9 and collagen II as described in Lanza, R. et al., Eds., Essentials of Stem Cell Biology, Academic Press, 2005; and Sive, J, I. et al., Mol Pathol., 2002, 55(2): 91-97.

Markers of cell type are assayed by methods known in the art illustratively including immunodetection methods such as immunofluorescence and Western blot; and nucleic acid detection methods such as RT-PCR and in situ hybridization. These and other methods for assays of specific cell type markers are described in detail in references cited herein and in standard references such as E. Harlow and D. Lane, Antibodies: A Laboratory Manual, Cold Spring Harbor Laboratory Press, 1988; F. M. Ausubel et al., Eds., Short Protocols in Molecular Biology, Current Protocols, Wiley, 2002; Ormerod, M. G., Flow Cytometry: a practical approach, Oxford University Press, 2000; Givan, A. L., Flow Cytometry: first principles, Wiley, New York, 2001; and J. Sambrook and D. W. Russell, Molecular Cloning: A Laboratory Manual, Cold Spring Harbor Laboratory Press, 3rd Ed., 2001.

The quantity of isolated cells admixed with an enzyme-treated donor tissue is generally in the range of about 10³-10⁹ cells/milliliter of the enzyme-treated donor tissue. Thus, in particular embodiments, a quantity of isolated MSCs admixed with an enzyme-treated donor tissue is in the range of about 10³-10⁹ cells/milliliter of the enzyme-treated donor tissue. In further particular embodiments, a quantity of isolated chondrocytes admixed with an enzyme-treated donor tissue is in the range of about 10³-10⁹ cells/milliliter of the enzyme-treated donor tissue.

The preferred cells that are added to the enzyme-treated donor tissue are autograft without cell culture. In additional embodiments, enzyme-treated donor tissue is autograft with cell culture, allograft with cell culture, and/or xenograft with cell culture. The enzyme-treated donor tissue may also be used without adding cells.

In preferred embodiments, isolated cells included in a transplant material of the present invention are isolated from the subject to be treated. In further embodiments, isolated cells included in a transplant material of the present invention are isolated from an individual of the same species as the subject to be treated. In still further embodiments, isolated cells included in a transplant material of the present invention are isolated from one or more individuals of a species other than the species of the subject to be treated.

Treatments of Intervertebral Discs

The normal human nucleus pulposus contains approximately 4 million cells per cubic millimeter. The invention seeks to regenerate an injured and/or diseased human nucleus pulposus in particular embodiments. Consequently, 4 million mesenchymal stem cells are added to each ml of proteoclycan-depleted nucleus pulposus tissue in certain embodiments of methods and compositions of the present invention. Alternatively 100,000; 200,000; 300,000; 400,000; 500,000; 600,000; 700,000; 800,000; 900,000 or less than 100,000 mesenchymal stem cells could be added per mi of proteoglycan-depleted nucleus pulposus tissue. Alternatively 1, 2, 3, 5, 6, 7, 8, 9, 10 or more than 10 million mesenchymal stem cells could be added to each ml of proteoglycan-depleted nucleus pulposus tissue.

Additional components are optionally included in a transplant material in addition to mesenchymal stem cells and enzyme-treated donor tissue. For example, culture media, collagen gels (particularly type II collagen gels), antibiotics, and or cytoidnes (including Platelet rich plasma, BMPs, etc) could be added to the transplant material in addition to mesenchymal stem cells and enzyme-treated donor tissue.

Such a component is optionally included before or during the administration of the transplant material to a subject.

The mesenchymal stem cells are ideally added to the enzyme-treated donor tissue at least three hours before administering the transplant material into a subject. Longer times may be used to increase the binding of mesenchymal stem cells to the enzyme-treated donor tissue and to begin to differentiate the mesenchymal stem cells.

Optionally, mesenchymal stem cells are cultured, at least partially, in the enzyme-treated donor tissue.

In a further option, the transplant material is placed into an environment that recreates the environment of the intervertebral disc (low ph, low oxygen, etc.). The external environment and the enzyme-treated donor tissue direct the mesenchymal stem cells to differentiate into nucleus pulposus cells. Pieces of enzyme-treated donor tissue could be added to the mesenchymal stem cells during the culture process. The therapeutic material, including cells and treated tissue, is administered, such as by injection, into the defective disc or discs of a subject.

The mesenchymal stem cells are preferably autologous (from the treated patient) in particular embodiments of the invention and the nucleus pulposus tissue is preferably xenograft (from a non-human animal).

Allograft or xenograft cells and allograft or autograft tissues may be used in other embodiments of the invention.

The therapeutic material could be gently washed with phosphate buffered saline to remove mesenchymal stem cells that are not bound firmly to the enzyme-treated donor tissue.

Collagen gels or other biocompatible materials including in-situ curing materials could be combined with the transplant materials. The in-situ curing materials could be added to help prevent the transplant material from leaking out of the patient's disc. The in-situ curing material could be injecting into the disc at the after injecting the transplant material to seal the opening in the AF.

Treatments for defects in articular cartilage

The isolated cartilage is treated with enzymes as described above to produce enzyme-treated cartilage having a reduced amount of at least one proteoglycan.

Chondrocytes and/or mesenchymal stem cells are combined with the modified cartilage having reduced proteoglycan content to produce a transplant material.

In particular embodiments, autologous chondrocytes and/or autologous MSCs are combined with the modified articular cartilage having reduced proteoglycan content. In addition to procedures for isolation and identification of chondrocytes described herein, autologous chondrocytes may also be obtained by sending pieces of cartilage tissue to Genzyme (Boston Mass., product known as Carticel). Autologous MSCs can be obtained as described in examples herein. The number of cells added to the modified articular cartilage having reduced proteoglycan content could be about the same concentration of cells per milliliter as normal articular cartilage tissue, or more or less cells per milliliter compared to normal articular cartilage tissue can be added.

Either mesenchymal stem cells or chondrocytes are added to the modified articular cartilage having reduced proteoglycan content. In further embodiments of methods and compositions of the present invention, both MSCs and chondrocytes are added to the modified articular cartilage having reduced proteoglycan content.

For example, cells added to the modified articular cartilage having reduced proteoglycan content are a combination of 90% MSCs and 10% cartilage cells or 80% MSCs and 20% cartilage cells, or 70% MSCs and 30% cartilage cells, or 60% MSCs and 40% cartilage cells, or 50% MSCs and 50% cartilage cells, or 40% MSCs and 60% cartilage cells, or 30% MSCs and cartilage cells, or 20% MSCs and 80% cartilage cells, or 10% MSCs and 90% cartilage cells, or less than 10% MSCs or less than 10% cartilage cells.

The subject's cartilage surrounding the defective region is optionally treated with enzymes to at least partially deplete the PGs of the cartilage that surrounds the defective region. The enzymes and concentrations listed above could be applied to the patient's cartilage that surrounds the defect. The enzymes are preferably applied to the cartilage at least several days before the therapeutic material is added to the defective region. Fibrin glue or other biocompatible material could be placed over the defective region of the cartilage after applying the enzymes to prevent the enzymes from leaking from the defective region of the cartilage. Enzymatic pre-treatment may improve the healing of the therapeutic material to the patient's cartilage. The therapeutic material could be placed in the defective region of the articular cartilage and covered with a periosteal flap as described it the procedure known as Autologous Chondrocyte Transplantation. Alternatively, the therapeutic material may be attached to patients' joints with alternative devices such as fibrin glue, bioresorbable tacks, sutures, or other devices.

Embodiments of the present invention may be used to combine MSCs or other cells with acellular autografts, allografts, or xenografts to treat other defective tissues in the body.

In a further embodiment of a method of transplantation of the present invention, a connective tissue in the body of the subject to receive the transplant is incubated with one or more proteoglycan-cleaving enzymes to reduce proteoglycan content of a connective tissue at or near the site of the transplant. Thus, for example, one or more proteoglycan-cleaving enzymes is administered to a diseased and/or injured intervertebral disc or at the site of a diseased and/or injured articular cartilage.

Embodiments of the invention described in this patent application may be used to direct mesenchymal stem cells or cartilage cells to form hyaline cartilage rather than the less desirable fibrocartilage. The proteins within the tissue and the exposed collagen network of the tissue direct MSCs to differentiate into cells similar the cells of the native tissue. The mechanical properties of the tissue are restored as the MSCs and cells that migrate into the tissue and replenish the PGs of the tissue.

According to embodiments of the invention, morsellized allograft, xenograft, and/or autograft intervertebral disc tissue is preferably treated with Chondroitinase ABC (C-ABC) to cleave the chondroitin sulfate and dermatan sulfate chains from the protein core of the proteoglycans (PGs) within the morsellized tissue. The enzymatic treatment reduces the PG content of the disc tissue but preserves the collagen fiber network of the particles of tissue. The PGs deep within the particles of tissue can thus be preserved.

A method of treating a defective tissue in a subject is provided which includes introducing a biomedical material of the present invention into a subject having a defective tissue, such as a defective intervertebral disc or defective articular cartilage. A biomedical material according to the present invention may include cells isolated from the individual to be treated or from one or more individuals of the same species. Such cells may also be isolated from one or more individuals of a different species than the subject. Cells may be cultured following isolation from the subject or others and prior to introduction into contact with the isolated tissue and/or introduction into the subject. Cells may be cultured, for instance, to expand the population of cells in order to increase the number of cells and/or to differentiate the cells.

A transplant material according to the present invention is administered to a subject at a site affected by connective tissue disease and/or injury in particular embodiments of the present invention. For example, TDM is injected into one or more defective intervertebral discs. The transplanted cells and the cells from the patient's disc replenish the proteoglycans within the processed tissue, bind the particles of tissue to each other, bind the particles of tissue to the surrounding disc tissue and increase the volume of the transplanted tissue.

The invention may be used to heal tears or clefts within the Nucleus Pulposus (NP), to heal tears or clefts within the Annulus Fibrosus (AF) or to heal defective regions of both the NP and the AF. The invention may also be used to increase the volume of the NP, the AF, or both. The patient's defective disc may be treated with C-ABC, or other enzyme, before injection of the TDM. Enzymatic treatment of the patient's disc before injection of the TDM may facilitate healing between the TDM and the patient's disc tissue.

A biomedical material of the present invention may be administered once or multiple times over an extended period of treatment.

The biomedical material is introduced into the subject in or near a region of the defective tissue in general. For example, the biomedical material is introduced into the nucleus pulposus, or annulus fibrosus, or both tissues of an intervertebral disc and/or at the site of diseased and/or damaged articular cartilage.

Disorders, diseases and injury affecting intervertebral discs, such as disc herniation and degenerative disc disease are well known in the art. Diseases, disorders and injury affecting articular cartilage are similarly well known in the art,

Thus, both indications for administration of compositions of biomedical material according to the present invention and techniques for assessment of these conditions and their improvement are both known and within the competence of one of ordinary skill in the art. Detailed description of such indications and assessment techniques can be found in standard reference texts such as Herzog, R. J., Magnetic Resonance Imaging of the Spine: Chapter 23, McMulloch, J. A., Microdiscectomy: Chapter 83, Krag, M. H., Spinal Fusion: Overview of Options and Posterior Internal Fixation Devices: Chapter 92 in Frymoyer, J. W (Ed.), The Adult Spine: Principles and Practice, Raven Press, 1991; and B. J. Cole and M. M. Malek, Eds., Articular Cartilage Lesions: A Practical Guide to Assessment and Treatment, Springer, 2004.

Administration of a biomedical material according to embodiments of the present invention includes delivery of a biomedical material including an enzyme-treated donor tissue to a site in a subject's body affected by a disease, disorder or injury capable of amelioration by the biomedical material. In particular embodiments, a biomedical material including an enzyme-treated donor tissue is delivered to a joint or other site typically characterized by the presence of articular cartilage in a healthy individual. In further particular embodiments, a biomedical material including an enzyme-treated donor tissue is delivered to an intervertebral disc.

Delivery of the biomedical material is by conventional techniques such as injection into the nucleus pulposus, and/or the annulus fibrosus. Detailed methods of delivering a composition to an intervertebral disc are described in detail in standard references such as Wallace, M. S., Human Spinal Drug Delivery: Methods and Technology, Chapter 14 in Yalcsh, T. L. (Ed.), Spinal Drug Delivery, Elsevier, 1999, and Mooney, V., Injection Studies: Role in Pain Definition, Chapter 25 in Frymoyer, J. W. (Ed.), The Adult Spine: Principles and Practice, Raven Press, 1991. Delivery to a joint may also be accomplished by standard techniques, such as injection into or near cartilage associated with the joint.

A commercial package for treatment of a defective tissue in a subject is provided according to the present invention which includes a quantity of a proteoglycan-cleaving enzyme and a quantity of isolated cells. Such cells are leukocytes, particularly monocytes; macrophages; platelets; cells derived from an intervertebral disc such as chondrocyte-like nucleus pulposus cells; fibrocytes; fibroblasts; mesenchymal stem cells; mesenchymal precursor cells; chondrocytes; or a combination of any of these. In preferred embodiments, mesenchymal stem cells, and/or chondrocytes are included in the commercial package. A commercial package optionally further includes a culture medium suitable for growth, maintenance and/or differentiation of the quantity of isolated cells. Such cells are optionally human or non-human isolated cells.

An embodiment of a commercial package for treatment of a defective tissue in a subject is provided according to the present invention which includes an enzyme-treated donor tissue. A quantity of isolated cells is further included in the package. Such cells are leukocytes, particularly monocytes; macrophages; platelets; cells derived from an intervertebral disc such as chondrocyte-like nucleus pulposus cells; fibrocytes; fibroblasts; mesenchymal stem cells; mesenchymal precursor cells; chondrocytes; or a combination of any of these. In preferred embodiments, mesenchymal stem cells, and/or chondrocytes are included in the commercial package. A commercial package optionally further includes a culture medium suitable for growth, maintenance and/or differentiation of the quantity of isolated cells. Such cells are optionally human or non-human isolated cells.

Embodiments of inventive compositions and methods are illustrated in the following examples. These examples are provided for illustrative purposes and are not considered limitations on the scope of inventive compositions and methods.

### EXAMPLES

### Example 1

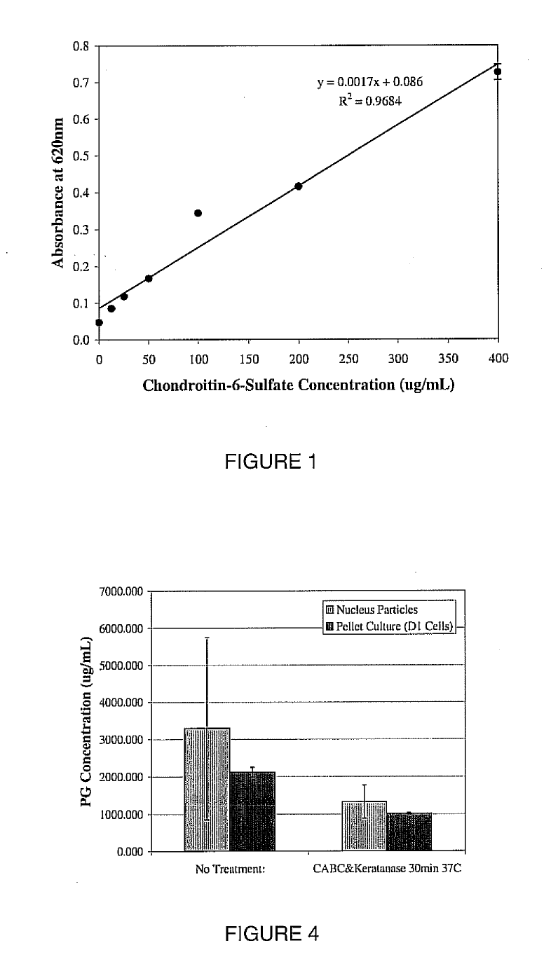

The nucleus pulposus (NP) and annulus fibrosus (AF) of goat intervertebral discs (IVDs) were separated and cut into 1-2 mm diameter pieces using surgical micro scissors. 25-100 microliter samples of NP or AF were placed into micro centrifuge tubes, and 0.8 ml of enzyme solution was added to each of these tubes. Six conditions were analyzed: solutions of 0.10 units/ml Chondroitinase-ABC, 0.10 units/ml Chondroitinase-ABC+0.10 units/ml Keratanase, or 3500 units/ml Hyaluronidase were added to the IVD samples for 15 minute and 30 minute time periods. After digestion, proteoglycan (PG) was extracted using 1 ml of 4 M Guanidine Hydrochloride (GuHCl) overnight. Alcian Blue was added to the PGs. Then PG-Alcian Blue was precipitated and centrifuged. The PG-Alcian Blue pellet was dissolved and the absorbance of the final solution was read at 620 nm using a spectrophotometer. The assay quantified only the sulfated PGs; Hyaluronan was not quantified. Chondriotin-6 sulfate with varying concentrations was used for calibration. It was found that the sulfated PG concentration varied linearly with absorbance, as shown in FIG. 1.

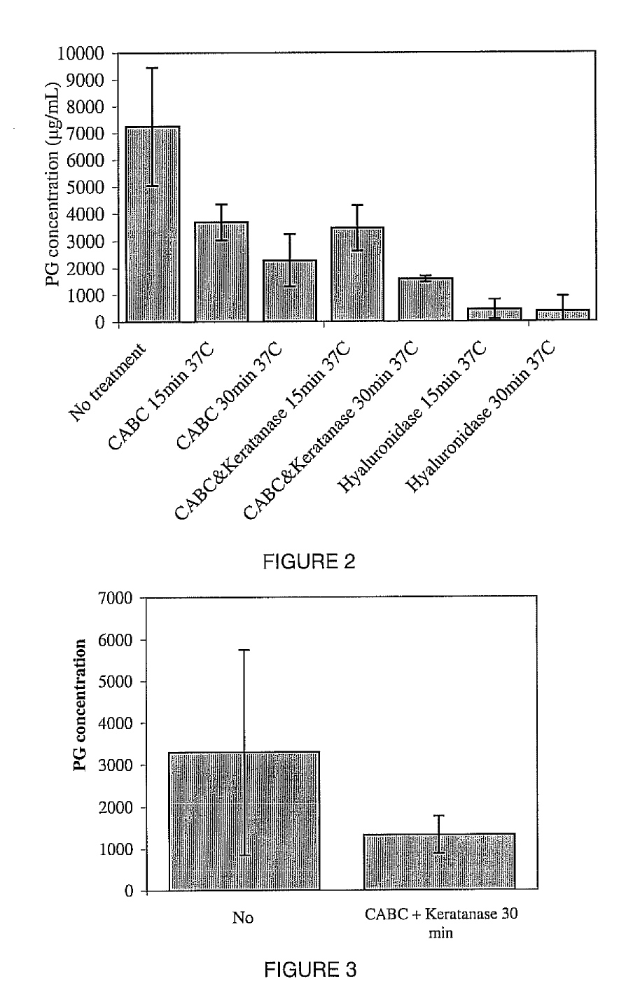

The results show that untreated NP particles had significantly more PGs than NP particles treated with Hyaluronidase, p=0.001, NP particles treated with CABC, p=0.0141, and NP treated with CABC+Keratanase, p=0.0061, as shown in FIG. 2.

The results from using AF particles show that AF particles treated with Hyaluronidase differed significantly from AF particles that were not treated with Hyaluronidase p<0.001. When comparing untreated AF to AF treated with CABC, p=0.0977, and comparing untreated AF to AF treated with CABC+Keratanase, p=0.0044.

### Example 2

The Nucleus Pulposus (NP) and Anulus Fibrosus (AF) of human intervertebral discs (IVDs) were separated and cut into 1-2 diameter particles using micro surgical scissors. The IVD particles were treated with enzymes, GuHCl, and Alcian Blue as described in Example 1. FIG. 3 shows the results of the treatment of these human IVD particles with CABC+Keratanase for 30 minutes

50,000 human mesenchymal stems (MSCs)/sample were added to the NP particles treated with enzymes CABC+Keratanase for 30 minutes, and grown in 1.804 mg/ml collagen gel. The cells and digested particles were incubated at 37° C. for 30 minutes, and then forced through a 16 GA needle. The cells and particles were then cultured for 48 hours after being forced through the needle. Analysis indicated that only a small number of cells, less than 10%, died.

### Example 3

Digested and Non-digested (Control) NP particles were placed into 15 ml centrifuge tubes, and one million MSCs were added to each tube. On day one the medium comprised of 0.5 ml DMEM with 10% FBS, 1% Penicillin Streptomycin, and 0.1% Amphotericin B. The NP particles, cells, and medium were centrifuged for 10 minutes at 3000 rpm and 37° C. The caps of the centrifuge tubes were slightly opened to allow oxygen flow, and the pellets were kept in an incubator. The medium, 0.5 ml DMEM with 10% FBS, 1% Penicillin Streptomycin, 0.1% Amphotericin B, and 10 ng/ml of TGF-Beta 1, was changed every two days. In order to determine PG content, at day 14 the pellet was washed with PBS and the tubes were vortexed to break down the pellet using GuHcl as described in example 1. PG content was determined with Alcian Blue assay also as described in example. In order to determine cell count, the pellet was washed with PBS and 0.5 ml medium without TGF-Beta 1 was added on day 14. The tubes were vortexed to break down the pellet and the mixture was then freeze-thawed three times for cell lysis. Cyto Tox assay was performed using a Cyto Tox 96 kit (Promega Corp. Madison, Wis.

Quantitation of proteoglycan content shows that control NP particles (non digested) lost 1180.454 ug/ml of PG concentration (35.77%), while the digested NP particles lost 310.997 ug/ml of PG concentration (20.73%) as illustrated in FIG. 4. Given the small number of samples tested the difference was not statistically significant. However, the data suggest the MScs may be producing PG or more PG when they are exposed to digested NP compared non-digested NP. The MSCs with the digested NP may be producing PGs in order to replace the PGs lost during the change of the culture media. The results of the cell assay show that when a graph of cells/50 microliter vs Absorbance at 492 nm is created a line with the equation y=861296-184254 results, with an r² value of 0.9598. Non digested NP particles had a mean of 100,000 MSCs attached to the NP particles and a maximum of 125,000 MSCs attached to the NP particles. The digested NP particles had a mean of 125,00 MSCs attached to the particles and a maximum of 250,000 MSCs attached to the particles. The differences were not statistically significant. However, the data suggest MSCs attach to the digested NP particles better than the non-digested NP particles. MSCs may also proliferate faster when cultured with digested NP particles than when cultures with non-digested NP particles. Additional tests will be done to quantify PG loss with culture media change and to study the effects of Platelet Rich Plasma.

### Example 4

In an in vivo Sheep study, 1-2 mm sheep IVD particles were digested with CABC+Keratanase for 30 minutes as described in example 1. The digested NP particles were frozen for future use, and when thawed they were washed with normal saline. Autologous Platelet Rich Plasma (PRP) was prepared from 50 to 100 ml of blood per sheep. The PRP was prepared with a COBE centrifuge in the standard fashion and the leukocytes were harvested and concentrated from the blood with the platelets. 9 drops of PRP and leukocytes and 1 drop of bovine thrombin were injected into a micro tube that contained 0.3 cc of digested NP sheep particles. The digested NP particles, platelets and cells were mixed and then injected into 2 IVDs per sheep. One sheep was sacrificed at each of the following time periods: 8 days, 14 days, and 28 days following surgery. Two sheep will be sacrificed at each of the following time periods: 56 days and 112 days following surgery. After gross examination of transected IVDs, the injected NP matrix+PRP+leukocyte mixture appears to be attached to the surrounding normal NP tissues. Ultimately, the specimens will be decalcified for histological examination.

### Example 5

Treatments for Defect in the Intervertebral Disc

IVDs are obtained from human donors or from animals. For example, IVDs may be obtained from herd-restricted populations of swine or cattle. The Nucleus Pulposus (NP) is separated from the Anulus Fibrosus (AF) of the IVD. For example, the NP can be aspirated from the IVDs through a 12-gauge needle. Alternatively, the NP can be excised from the IVDs with a knife or motorized processor. The NP tissue is cut into pieces approximately 1×1 mm in size or smaller. Alternatively, the NP may be cut into pieces 2×2 mm, 3×3 mm, or 4×4 mm in size or larger. The pieces of NP can be spherical or other shapes. The NP tissue is frozen at −70 degrees C. before or after mincing the tissue. The minced NP tissue is treated with C-ABC, keratanase I, Iceratanase II, Hyaluronidase, and/or other enzyme or enzymes that selectively cleave PGs. For example, the particulated NP tissue may be treated with C-ABC (0.25 IU/ml for 90 minutes at 37 degrees C.) or hyaluronidase (3,500 units/ml for thirty minutes). Alternatively, the NP tissue may be treated with C-ABC (0.1 units/mi, ICN Biochemicals), keratanase I (0.1 units/ml, Sigma), and keratanse II (0.001 units/ml, AMS Biotechnology, Whitney, UK) for three hours at room temperature. The particles of NP tissue should be completely submerged in the enzymatic solutions. Alternative dosages of enzymes and alternative treatment periods could be used. For example, the NP tissue could be treated with 1 IU/ml, 0.5 IU/ml, 0.25 IU/ml, 0.1 IU/ml, 0.05 IU/ml, 0.01 IU/ml, 0.005 IU/ml, 0.001 IU/ml, 0.005 IU/ml, 0.0001 IU/mI or less C-ABC for 30 minutes, 60 minutes, 2 hours, 3 hours, 4, hours, 5 hours, 6 hours, 7 hours, 8 hours, 10 hours, 12 hours, or more and/or 1.0 units/ml, 0.5 units/ml, 0.4 units/ml, 0.3 units/ml, 0.2 units/ml, 0.08 units/ml, 0.06 units/ml, 0.04 units/ml, 0.02 units/ml, 0.01 units/ml, 0.005 units/ml, 0.001 units/ml, 0.0005 units/ml, or 0.0001 units/ml of keratanase I for similar time periods and/or 0.01 units/ml, 0.008 units/ml, 0.006 units/ml, 0.004 units/ml, 0.002 units/ml, 0.0008 units/lm, 0.0006 units/ml, 0.0004 units/ml, 0.0002 units/ml, 0.00001 units/ml or less keratanase II for similar time periods.

### Example 6

Allograft or xenograft Nucleus Pulposus (NP) can be frozen to −70° C. A grinder-like instrument may be used to dice the tissue into small particles; for example, on the order of 1 mm in diameter. The tissue could be thawed then completely submerged in a tube containing one milliliter phosphate buffered saline and 0.5 U Chondroitinase-ABC (Sigma, Poole, UK) for three hours at 37° C. The treated tissue is then washed with phosphate buffered saline.

Ten milliliters (or other amount) of marrow aspirate (obtained by combining five, two milliliter marrow aspirates from different locations in the iliac crest or spine) is added to the tissue. Heparin may be added to the marrow aspirate to prevent coagulation of the aspirate. The mixture of processed tissue and marrow aspirate is stirred or agitated at 37° C. for 30 minutes (or other duration). The material is strained through a filter with pores smaller than 0.5 mm in diameter. One to two milliliters of the liquid that massed through the filter may be added to the material that is removed from the filter.

The therapeutic disc material above the filter is comprised primarily of processed disc tissue and cells from the marrow, including mesenchymal stem cells (MSCs) and mononuclear cells. The TDM is injected into defective discs through an 18-gauge needle. Additional therapeutic materials including platelet rich plasma (PRP) or cytokines including BMP-2 (InFuse, Medtronic Sofamor Danek) or OP-1 (Stryker) may added to the TDM.

The patient's disc can be treated with 1 U or less of C-ABC three weeks before injecting the TDM into the treated disc. The material is preferably injected into defective regions of the disc including clefts in the NP and Annulus Fibrosus (AF). The location of such defects may be detected with CT discograms or Ultrasound. Different amounts of C-ABC (e.g. 0.9 U, 0.8 U, 0.7 U, or less or 1.1 U, 1.2 U, 1.3 U, or more) may be used in alternative embodiments of the invention. Different periods of time between injection of C-ABC and the injection of the TDM (e.g. 2 weeks, 1 week, or less or 4 weeks, 5 weeks or more) may be used in other embodiments of the invention. Different enzymes may be injected into the patient's disc prior to injecting the TDM in alternative embodiments of the invention. For example, the patient's disc could be injected with 1000 U (or 500 U, 2,000 U, 3,000 U or other amount) Chymopapin four weeks prior to injection of the TDM.

Different concentrations of C-ABC and/or different processing times may be used in alternative embodiments of the invention. For example, the PG of the processed tissue may be increased by reducing the concentration of the C-ABC (e.g. 0.4 U, 0.3 U, 0.2 U, 0.1 U, or less) and/or decreasing the processing time (e.g. 2 hours, 1 hour, 0.9 hour, 0.8 hour, 0.7 hour, or less), and/or increasing the size of the pretreated particles of disc material (e.g. 2 mm diameter, 3 mm diameter, 4 mm diameter, 5 mm diameter, or larger). Conversely the PG of the processed tissue may be decreased by increasing the concentration of the C-ABC (e.g. 0.6 U, 0.7 U, 0.8 U, 0.9 U, or higher) and/or increasing the processing time (e.g. 4 hours, 5 hours, 6 hours, 7 hours, or more) and/or decreasing the size of the pretreated particles of disc material (e.g. 0.9 mm diameter, 0.8 mm diameter, 0.7 mm diameter, 0.6 mm diameter, or less). An intact donor disc may be processed as described above without cutting the disc into pieces prior to processing the tissue in alternative embodiments of the invention. Alternative the donor disc can be cut into elongate pieces or other shaped pieces prior to treating the tissue.

Donor AF disc tissue may be treated in a similar manner. Such TDM could be used to treat defective regions of patient's AF. Alternatively, various combinations of donor AF and donor NP could be processed together and injected together to treat disc disease.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

Having described my invention, I claim:

1. A biomedical material, comprising:
an enzyme-treated isolated donor tissue, the enzyme-treated donor tissue characterized by a reduced amount of at least one type of proteoglycan compared to untreated tissue.

2. The biomedical material of claim 1, further comprising a quantity of isolated cells in contact with the enzyme-treated isolated donor tissue.

3. The biomedical material of claim 2, wherein the isolated cells are cells selected from the group consisting of: leukocytes; monocytes; macrophages; platelets; intervertebral disc-derived cells; chondrocyte-like nucleus pulposus cells; fibrocytes; fibroblasts; mesenchymal stem cells; mesenchymal precursor cells; chondrocytes; and a combination of any of these.

4. The biomedical material of claim 1, wherein the enzyme-treated isolated donor tissue is a non-human tissue.

5. The biomedical material of claim 1, wherein the enzyme-treated isolated donor tissue is a human tissue.

6. The biomedical material of claim 1, wherein the enzyme-treated isolated donor tissue is articular cartilage.

7. The biomedical material of claim 1, wherein the enzyme-treated isolated donor tissue is an intervertebral disc tissue selected from the group consisting of: nucleus pulposus tissue; annulus fibrosis tissue; and a combination thereof.

8. The biomedical material of claim 1, wherein the enzyme-treated isolated donor tissue is substantially free of intact, living cells endogenous to the enzyme-treated isolated donor tissue.

9. The biomedical material of claim 1, wherein the amount of at least one type of proteoglycan is reduced by 1-100% compared to an untreated tissue.

10. The biomedical material of claim 2, wherein the quantity of isolated cells is in the range of about 10³-10⁹ cells/milliliter of the enzyme-treated donor tissue.

11. The biomedical material of claim 1, wherein the enzyme-treated donor tissue is in the form of particles.

12. The biomedical material of claim 1, wherein the particles have an average particle size in the range of about 0.01 mm³-30 mm³, inclusive.

13. A method of treating a defective tissue in a subject, comprising:
introducing a biomedical material of claim 1 into a subject having a defective tissue.

14. The method of claim 13, wherein the defective tissue is an intervertebral disc.

15. The method of claim 13, wherein the defective tissue is articular cartilage.

16. A method of treating a defective tissue in a subject, comprising:
introducing a biomedical material of claim 2 into a subject having a defective tissue.

17. The method of claim 16, wherein the quantity of isolated cells is isolated from the subject.

18. The method of claim 16, wherein the quantity of isolated cells comprises cells isolated from an individual of the same species as the subject.

19. The method of claim 15, wherein the biomedical material is introduced into the subject in or near a region of the defective tissue.

20. A method of tissue transplantation, comprising:
providing tissue to be transplanted;
using an enzyme to at least partially digest proteoglycans in the tissue while at least partially preserving the collagen network of the tissue; and
transplanting the treated tissue into a recipient.

21. The method of claim 20, wherein the tissue is allograft, xenograft, and/or autograft tissue.

22. The method of claim 20, wherein the tissue is treated with a proteoglycan-cleaving enzyme to reduce the proteoglycan content of the tissue by 1-100% compared to an untreated tissue.

23. The method of claim 20, including adding stem cells to the tissue.

24. The method of claim 20, wherein the tissue is connective tissue.

25. The method of claim 20, wherein the transplanted tissue is used to heal tears or clefts within an intervertebral disc structure selected from the group consisting of: a nucleus pulposus; an annulus fibrosus; and a combination thereof.

26. The method of claim 20, wherein the transplanted tissue is used to increase the volume of an intervertebral disc structure selected from the group consisting of: a nucleus pulposus; an annulus fibrosus; and a combination thereof.

27. The method of claim 20, wherein the tissue is treated with a proteoglycan-cleaving enzyme selected from the group consisting of: a chondroitinase, a hyaluronidase, a keratanase and a combination thereof.

28. The method of claim 20, wherein the tissue is treated with a proteoglycan-cleaving enzyme is an enzyme which digests polysaccharide side chains in a proteoglycan without digesting the protein portion of the proteoglycan.

29. The method of claim 20, wherein the tissue is treated with an enzyme selected from the group consisting of chymopapain, collagenase, cathepsin B, cathepsin G, calpain I, and a combination thereof.

30. The method of claim 20, wherein the tissue is treated with a second material that deactivates the enzyme.

31. The method of claim 20, wherein the tissue is treated with alpha-2-macroglobulin or a cathepsin before transplanting the treated tissue into a recipient.

32. The method of claim 20, wherein the tissue is derived from, or forms part of, articular cartilage or an intervertebral disc.

33. A commercial package for treatment of a defective tissue in a subject, comprising:
an enzyme-modified isolated donor tissue.

34. The commercial package of claim 33, wherein the enzyme-modified isolated donor tissue is enzyme-modified isolated donor connective tissue.

35. The commercial package of claim 33, further comprising a quantity of cells admixed with the enzyme-modified isolated donor tissue.
